(19) United States
(12) Patent Application Publication
(10) Pub. No.: US 2008/0160065 A1
(43) Pub. Date: Jul. 3, 2008

(54) DRUG DELIVERY POLYMER WITH HYDROCHLORIDE SALT OF CLINDAMYCIN

(76) Inventors: Janet Anne HALLIDAY, West Lothian (GB); Denis Andrew CARR, Glasgow (GB); Lynn BOYD, Hamilton (GB); Monica MACGREGOR, Cambuslang (GB); Audrey THOM, Hamilton (GB); Linda KELLY, Glasgow (GB); Mark Alexander LIVINGSTONE, Irvine (GB); Lilias Morton CURRIE, Blantyre (GB)

Correspondence Address: Law Office of John K. Pike, PLLC, 2121 Eisenhower Avenue, Suite 200, Alexandria, VA, 22314, US

(21) Appl. No.: 11/777,175

(22) Filed: Jul. 12, 2007

Related U.S. Application Data
(60) Provisional application No. 60/807,149, filed on Jul. 12, 2006.

(51) Int. Cl. A61K 9/02; A61K 9/00; A61K 31/4025; A61P 31/04; A61P 31/10

(52) U.S. Cl. 424/433; 424/435; 424/436; 514/422; 424/484

(57) ABSTRACT

One embodiment provides an insert, which includes a non-degradable hydrogel matrix and clindamycin hydrochloride in contact with the matrix, wherein the insert is suitable for mammalian intravaginal, buccal, or intrarectal use. Methods of using and making the insert are also provided.

### BRIEF DESCRIPTION OF THE DRAWINGS

FIG. 1 shows drug release and stability data according to an exemplified embodiment.

### DETAILED DESCRIPTION OF THE SEVERAL EMBODIMENTS

One embodiment provides an insert, which comprises a non-degradable hydrogel matrix and clindamycin hydrochloride in contact with the matrix, wherein the insert is suitable for mammalian intravaginal, buccal, or intrarectal use.

One embodiment provides a method, which comprises contacting a mammalian vagina, buccal cavity, or rectum with an insert, which comprises a non-degradable hydrogel matrix and clindamycin hydrochloride in contact with the matrix, wherein the insert is suitable for mammalian intravaginal, buccal, or intrarectal use.

One embodiment provides a method, which comprises contacting clindamycin hydrochloride with a non-degradable hydrogel matrix.

One embodiment provides a package, which comprises an insert, which comprises a non-degradable hydrogel matrix and clindamycin hydrochloride in contact with the matrix, wherein the insert is suitable for mammalian intravaginal, buccal, or intrarectal use, and at least one packaging material surrounding the insert.

One embodiment provides a retrievable device, which comprises an insert, which comprises a non-degradable hydrogel matrix and clindamycin hydrochloride in contact with the matrix, wherein said insert is suitable for mammalian intravaginal, buccal, or intrarectal use, and a device in contact with the insert and adapted to retrieve the insert from a vagina or rectum.

One embodiment provides an insertable device, which comprises an insert, which comprises a non-degradable hydrogel matrix and clindamycin hydrochloride in contact with the matrix, wherein said insert is suitable for mammalian intravaginal, buccal, or intrarectal use, and a device in contact with the insert and adapted to insert the insert into a vagina or rectum.

One embodiment provides a method for inhibiting a microorganism. The method includes contacting a microorganism with an effective amount of a composition that includes clindamycin hydrochloride in a hydrogel matrix, for a period of time effective to inhibit the microorganism.

One embodiment provides a method for treating bacterial vaginosis in a human patient. The method includes oral, intrarectal, and/or intravaginal administration to a patient in need of such treatment an effective amount of a composition that includes clindamycin hydrochloride in a hydrogel matrix.

One embodiment relates to the therapeutic practice of introducing into an afflicted vagina, or orally, or intrarectally a therapeutically effective amount of a formulation of clindamycin hydrochloride in a hydrogel matrix. One embodiment relates to the prophylactic practice of introducing the clindamycin hydrochloride in a hydrogel matrix for preventing bacterial vaginosis in human female patients that are at risk or susceptible to it. To that end, a prophylactic amount of an insert, which includes a hydrogel matrix and clindamycin hydrochloride may be suitably administered intravaginally, intrarectally, or orally chronically or for a time period while the susceptibility exists.

One embodiment relates to a method for treating or preventing one or more of bacterial vaginosis, pelvic inflammatory disease, endometritis, post-operative infection following gynecologic surgery, pre-term labor, pre-term birth, urinary tract infection, recurrent urinary tract infection, upper genital tract infection, postpartum endometritis, post-hysterectomy infection, post-miscarriage infection, and post-abortion infection, which includes using or administering clindamycin hydrochloride in contact with a hydrogel polymer.

One embodiment relates to a method for improving success rates for artificial insemination/fertility treatment, which includes using or administering clindamycin hydrochloride in contact with a hydrogel polymer.

One embodiment provides an intravaginal, buccal, or intrarectal insert that delivers a minimum effective dose of clindamycin hydrochloride.

As used in this specification and the appended claims, the singular forms “a,” “an” and “the” include plural referents unless the context clearly dictates otherwise. Thus, for example, reference to “an active agent” includes a single active agent as well two or more different active agents in combination.

The terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The term, “hydrophilic” is used herein in its conventional sense, meaning having a strong tendency to attract, adsorb and/or absorb water and/or to swell in the presence of water, aqueous solutions or mixtures, and/or bodily fluids.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. “Treating” a patient by administering a beneficial agent includes prevention of a particular disorder or unwanted physiological event as well as treatment of a clinically symptomatic individual by inhibiting or causing regression of a disorder or disease.

By the term “effective amount” of a therapeutic agent is meant a nontoxic but sufficient amount of a beneficial agent to provide the desired effect. The amount of beneficial agent that is “effective” may vary from subject to subject, depending on the age and general condition of the individual, the particular beneficial agent or agents, and the like. Thus, it is not always possible to specify an exact “effective amount.” However, an appropriate “effective” amount in any individual case may be determined by one of ordinary skill in the art using routine experimentation and given the teachings herein.

The term “controlled release” refers to a formulation, dosage form, or region thereof from which release of a beneficial agent is not immediate, i.e., with a “controlled release” dosage form, administration does not result in immediate release of the beneficial agent in an absorption pool. The term is used interchangeably with “nonimmediate release” as defined in Remington: The Science and Practice of Pharmacy, Nineteenth Ed. (Easton, Pa.: Mack Publishing Company, 1995), the entire contents of which being hereby incorporated by reference. In general, the term “controlled release” as used herein includes sustained release and delayed release formulations. One embodiment includes a controlled release insert, which contains at least clindamycin hydrochloride in contact with a hydrogel matrix, and optionally, a control release agent, for example, a coating.

The term “sustained release” (synonymous with “extended release”) is used in its conventional sense to refer to a formulation, dosage form, or region thereof that provides for gradual release of a beneficial agent over an extended period of time, and that preferably, although not necessarily, results in substantially constant blood and/or localized levels of the agent over an extended time period. One embodiment includes a sustained release insert, which contains at least clindamycin hydrochloride in contact with a hydrogel matrix. One or more release agents may be present, for example, a co-solute, swelling agent, or the like.

The term “unit dose” or “unit dosage form” as used herein refers to physically discrete units of such composition suitable for use as unitary dosages by mammalian subjects. Each unit contains a predetermined quantity of clindamycin hydrochloride calculated to produce the desired therapeutic and/or prophylactic effect in association with the hydrogel matrix.

The term “biocompatible” refers to a material that is not biologically undesirable, i.e., the material may be incorporated into a formulation administered to a patient generally without resulting in substantial undesirable biological effects. In one embodiment, the insert and/or hydrogel matrix is biocompatible.

The term “pharmaceutically acceptable,” as in a “pharmaceutically acceptable” carrier or excipient refers to a carrier or excipient that has met the required standards of toxicological and manufacturing testing or that it is included on the Inactive Ingredient Guide prepared by the U.S. Food and Drug administration. In one embodiment, the insert and/or hydrogel matrix is pharmaceutically acceptable.

“Pharmacologically active” (or simply “active”) as in a “pharmacologically active” derivative or analog, refers to a derivative or analog having the same type of pharmacological activity as the parent compound and preferably, but not necessarily, approximately equivalent in degree.

The term “polymer” as used herein refers to a molecule containing a plurality of covalently attached monomer units, and includes branched, dendrimeric and star polymers as well as linear polymers. The term also includes both homopolymers and copolymers, e.g., random copolymers, block copolymers and graft copolymers, as well as uncrosslinked polymers and slightly to moderately to substantially crosslinked polymers.

The term “vagina” or “intravaginal” as used herein is intended to be inclusive of the vaginal region generally, including also the vulva and the cervix. Also, the term “afflicted vagina” as used herein is intended to be inclusive of bacterial vaginosis (BV) and any other indication described herein.

The term “rectum” or “intrarectal” as used herein is intended to include the terminal portion of the large intestine extending from about the descending and/or sigmoid colon through the anal canal.

The term, “oral”, “mouth cavity” and “buccal” as used herein are intended to include the mouth.

The term, “body cavity” is intended to include any of the vagina, rectum, or mouth, singly or collectively.

The term, “non-degradable” as in “non-degradable” hydrogel matrix, is intended to mean that the hydrogel matrix does not degrade during intended or normal use, e.g., in the vagina, mouth, or rectum.

Clindamycin hydrochloride (7(S)-Chloro-7-deoxylincomycin hydrochloride; 7-Chloro-7-deoxylincomycin hydrochloride; L-threo-alpha-D-galacto-octopyranoside, methyl 7-chloro-6,7,8-trideoxy-6-((((2S,4R)-1-methyl-4-propyl-2-pyrrolidinyl)carbonyl)amino)-1-thio-, monohydrochloride; (2S-trans)-Methyl 7-chloro-6, 7, 8-trideoxy-6-[[(1-methyl-4-propyl-2-pyrrolidinyl)carbonyl]amino]-1-thio-L-threo-α-D-galacto-octopyranoside hydrochloride monohydrate) is a known compound. It is the hydrochloride salt of clindamycin. In one embodiment, clindamycin hydrochloride is a semi synthetic lincosamide antibiotic, which may be produced by a three stage method of fermentation followed by chlorination and reaction with hydrochloric acid.

In one embodiment, the structure of clindamycin hydrochloride may be depicted as follows:

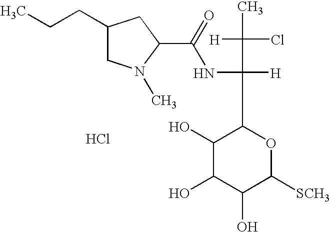

In one embodiment, the structure of clindamycin hydrochloride may be depicted as follows:

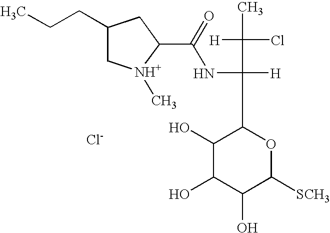

In one embodiment, the structure of clindamycin hydrochloride may be depicted as follows:

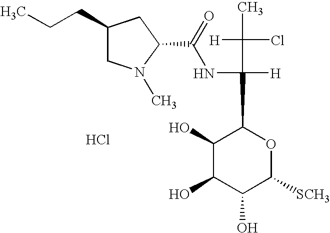

In the structure above, one stereoisomer of clindamycin hydrochloride is shown, but other stereoisomers are possible. All stereoisomers of clindamycin and clindamycin hydrochloride are contemplated herein. In one embodiment, the active agent is the clindamycin free base. Clindamycin has been used for several decades as a broad-spectrum antibiotic that has activity against gram-positive and Gram-negative aerobic and anaerobic bacteria, together with activity against Leptospira spp., Mycoplasma spp., and protozoa. The antibacterial activity of clindamycin is dependent on the susceptibility of the pathogen, measured as the minimal inhibitory concentration (MIC) and the serum or body fluid concentration of antibiotic. The MIC for susceptible Gram-positive cocci are 0.002-0.8 mg/l, and for most strains of Bacteriodes≦2 mg/l.

Bacterial vaginosis (“BV”) is one of the most common causes of vaginal discharge and is believed to be caused by an imbalance of the microbial flora. One or more of the microorganisms, Bacteroides fragilis, Gardnerella vaginalis, Mobilincus spp. are believed to be responsible for bacterial vaginosis. A clinical diagnosis of BV may be made if two or more of the following four clinical criteria are present: (1) a homogenous discharge; (2) a pH≧4.7; (3) a “fishy” amine odor upon the addition of 10% KOH to discharge; (4) presence of epithelial clue cells representing greater than or equal to 20% of vaginal epithelial cells.

Vaginal infection with G. vaginalis has been associated with possible sequelae, such as pelvic inflammatory disease, endometritis, and premature labor that have an attendant, significant morbidity profile. Although there is no direct evidence linking BV with these conditions, it is not unreasonable to assume that an overgrowth of 10,000 to 100,000 anaerobic organisms in the vagina may result in certain genital diseases. In the last decade there has been a tendency towards a reduction in gonorrhea and trichomoniasis while, during the same time span, there has been an increase in the so called “non-specific genital disease.” BV may account for significantly more total vaginitis patients than either Candida or trichomoniasis.

Clindamycin binds to the 50S subunit of bacterial ribosomes, inhibiting protein synthesis. It shows activity against pneumococci and is active against many strains of S. aureus. Clindamycin is active against anaerobes, especially B. fragilis, also Mobiluncus spp., Gardenerella spp., and Atobopium spp. The drug also shows some activity towards atypical organisms or parasites such as Chlamydia spp., Toxoplasma gondii and some Plasmodium species and strains.

Numerous studies have been conducted with oral and vaginal products that demonstrate the efficacy of clindamycin in bacterial vaginosis. Clindamycin is currently one of the two standard treatments for this condition, the other being metronidazole. Intravaginal application of clindamycin has been shown to be clinically effective in the treatment and prevention of BV.

When clindamycin is given parenterally, it is hydrolysed in vivo to the active clindamycin. When injected intramuscularly, the peak plasma concentration is not attained until 2 hours in adults and 1 hour in children. These values are approximately 61 μg/ml after a 300 mg dose and 9 μg/ml after a 600 mg dose, respectively (Goodman and Gilman's The Pharmacological Basis of Therapeutics, Ninth Edition (Hardman, J. G. et al, eds.) McGraw-Hill, New York, 1990).

For currently available vaginal compositions, the extent of absorption after vaginal administration depends on the formulation. For the 2% vaginal cream formulations (e.g. Dalacin® cream 2%), peak plasma levels after daily dosing of 100 mg clindamycin (i.e. 5 grams of the 2% cream each day) averaged 20 ng/ml (range 3-93 ng/ml). In women with BV, the amount of clindamycin absorbed after use of Dalacin® cream (2%) is reported to be 4% of the administered dose (Pharmacia Limited SPC for Dalacin® SmPC, July 2002). Studies with clindamycin phosphate vaginal suppositories (CLEOCIN™ vaginal ovules) containing 100 mg clindamycin showed that approximately 30% (range 6 to 70%) of the administered dose was absorbed into the systemic circulation, based on AUC data compared against the AUC after a sub-therapeutic 100 mg intravenous dose given in the same volunteers (Pharmacia and Upjohn Company; SPC for CLEOCIN™ vaginal ovules, January 2003).

Methods of preparing both clindamycin and clindamycin hydrochloride are known, for example, from U.S. Pat. Nos. 3,487,068 and 4,895,934, the entire contents of each of which being independently incorporated herein by reference.

Indications for which the insert is effective include bacterial vaginosis, pelvic inflammatory disease, endometritis, post-operative infection following gynecologic surgery, pre-term labor, pre-term birth, improving success rates for artificial insemination/fertility treatment, prophylaxis prior to vaginal gynecologic surgery, urinary tract infection, recurrent urinary tract infection, upper genital tract infection, postpartum endometritis, post-hysterectomy infection, post-miscarriage infection, and post-abortion infection.

One embodiment of a hydrogel is a three-dimensional network of hydrophilic polymer chains that are crosslinked through either chemical bonding, physical bonding, or a combination thereof. In a chemical hydrogel, the polymer chains are crosslinked directly or indirectly to each other by covalent bonds. In a physical hydrogel, the polymer chains are crosslinked directly or indirectly to each other by physical bonds, such as ionic bonds, hydrogen bonds, Van der Waals interactions, and the like. Combination hydrogels may be crosslinked via a combination of chemical and physical bonds.

In one embodiment, the hydrogel is completely or substantially completely crosslinked. In one embodiment, when the hydrogel is completely crosslinked, it is one molecule regardless of its size. In one embodiment, the hydrogel is insoluble in all solvents at elevated temperatures under conditions where polymer degradation does not occur. In one embodiment, the hydrogel is insoluble in aqueous solvents at elevated temperatures under conditions where polymer degradation does not occur.

Because of the hydrophilic nature of the polymer chains, hydrogels absorb water, with the result that the hydrogel matrix swells. In one embodiment, the hydrogel swells in response to contact with a bodily fluid, such as a vaginal fluid, saliva, and/or rectal fluid.

Suitable hydrogels are described in U.S. Pat. Nos. 5,017,382; 4,931,288; 4,894,238; and 6,488,953, the entire contents of which being independently hereby incorporated by reference.

In one embodiment, in the unswollen state, the hydrogel matrix is a solid or is substantially non-deformable. Here, the term solid is intended to distinguish the hydrogel matrix from a sol, sol-gel, gel emulsion, or colloid, which have a lower degree of crosslinking, a lower degree of gelation, a higher concentration of uncrosslinked or soluble polymers, and/or are more easily deformed in the non-swollen state.

In one embodiment, the hydrogel matrix has a gel to sol ratio (the gel being the insoluble, crosslinked, polymer fraction, and the sol being the soluble, uncrosslinked, polymer fraction) of 75:25 by weight or more. This range includes all values and subranges therebetween, including, for example, gel:sol ratios of 75:25, 80:20, 85:15, 90:10, 91:9, 92:8, 93:7, 94:6, 95:5, 96:4, 97:3, 98:2, 99:1, 99.1:0.9, 99.2:0.8, 99.3:0.7, 99.4:0.6, 99.5:0.5, 99.6:0.4, 99.7:0.3, 99.8:0.2, 99.9:0.1, and 100:0.

The hydrogel matrix may be a thermoset, elastomer, thermoplastic elastomer, crosslinked polyethylene oxide, crosslinked polyethylene glycol, urethane, copolymers thereof, and interpenetrating polymer networks thereof.

In one embodiment, the hydrogel matrix includes polyethylene glycol crosslinked with urethane. In one embodiment, the hydrogel matrix includes a polyethylene glycol crosslinked with 1,2,6 hexanetriol and dicyclohexylmethane 4,4′-diisocyanate as a chain extender and ferric chloride as a catalyst.

The hydrogel matrix is non-degradable, meaning that it does not degrade during intended or normal use, e.g., in the vagina, mouth, or rectum. As such, the insert should be distinguished from an ovule, suppository, or pessary, which are designed to degrade during normal use, i.e., they release their contents mainly through biodegradation, erosion, dissolution, dissociation, hydrolysis or other degradation of the matrix material.

The dimensions of the dry hydrogel matrix may suitably range from about 10 to 50 mm in length, about 1 to 20 mm in width, and about 0.5 to 10 mm in thickness. These ranges include all values and subranges therebetween, including, for example, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.75, 3, 4, 5, 6, 7, 8, 9, 10, 11, 12, 13, 14, 15, 16, 17, 18, 19, 20, 25, 30, 40, and 50 mm as appropriate, and any combination thereof.

The weight of the blank hydrogel matrix may suitably range from about 100 to 1000 mg. This range includes all values and subranges therebetween, including, for example, 100, 101, 102, 103, 104, 105, 106, 107, 108, 109, 110, 115, 120, 130, 140, 150, 160, 170, 180, 190, 200, 225, 250, 275, 300, 325, 350, 375, 400, 425, 450, 475, 500, 525, 550, 575, 600, 700, 800, 900, 1000 mg, and any combination thereof.

Clindamycin hydrochloride is in contact with the hydrogel matrix, meaning that it is absorbed or dispersed throughout the entirety or a portion of the matrix hydrogel, is suspended in a portion or throughout the entirety of the matrix hydrogel, is coated on one or more surfaces of the matrix hydrogel, or a combination thereof. In one embodiment, when in normal use, the matrix swells via uptake of a liquid or bodily fluid such as, for example, vaginal fluid, saliva, bodily fluid, rectal fluid, and the like, and clindamycin hydrochloride, clindamycin free base, or both, is released from the matrix.

The quantity of clindamycin hydrochloride introduced intravaginally, intrarectally, or orally as a single or unit dose can vary widely, depending upon many variables, such as the age and physical condition of the patient, the extent of the patient's affliction, the nature of the patient's affliction, the duration of administration, the frequency of administration, the need for prophylaxis, the need for therapeutic administration, the release rate of active agent, and the like.

The quantity of active agent in a unit dose is generally at least about 1 milligram (mg), and is not more than about 500 mg. This range includes all values and subranges therebetween, including, for example, 1, 2, 3, 4, 5, 6, 7, 8, 9, 10, 12, 14, 16, 18, 20, 30, 40, 50, 60, 70, 80, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 250, 300, 350, 400, 500 mg, and any combination thereof. The unit dose may be measured in terms of the amount of clindamycin hydrochloride contained in the hydrogel matrix or the amount of clindamycin base delivered, noting that 100 mg clindamycin=108.5658 mg clindamycin hydrochloride. For example, a 100 mg clindamycin unit dose insert would contain 108.5658 mg clindamycin hydrochloride. In one embodiment, one insert contains clindamycin hydrochloride in an amount equivalent to 100 mg clindamycin.

The clindamycin hydrochloride may be present in the hydrogel matrix in an amount ranging from about 5 to 75% w/w hydrogel matrix. Here, the “% w/w hydrogel matrix” is based on the weight of the clindamycin hydrochloride relative to the weight of the blank hydrogel matrix. This range includes all values and subranges therebetween, including, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 47, 49, 50, 55, 60, 70, and 75% w/w hydrogel matrix and any combination thereof.

The inserts may be administered orally, intrarectally, and/or intravaginally once or more than once as appropriate. If administered more than once, the inserts may be administered on a regular basis or on an irregular basis. The insert may be administered at a rate of one to four times over a time period ranging from a single day to one year, optionally repeating as necessary, and optionally with one or more intervals of non-administration. These ranges include all values and subranges therebetween, including, for example, 1, 2, 3, and 4 times for administration, and a time period of 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 days, and 1, 2, 3, 4, 5, 6, 7, 8, 9, 10, 11, and 12 months, and any combination thereof.

In one embodiment, the inserts may be administered in connection with a pregnancy or planned or unplanned pregnancy. For example, in one embodiment, the inserts may be administered at any time before conception to delivery and thereafter. Some examples of administration times related to pregnancy include 1, 2, or 3 months before conception, conception, 1, 2, 3, 4, 5, 6, 7, 8 and 9 months after conception, during gestation, delivery, and post-partum.

The total daily dose may suitably range from about 1 mg to about 1500 mg, which range includes all values and subranges therebetween, including, for example, 1, 2, 3, 4, 5, 6, 7, 8, 9, 10, 12, 14, 16, 18, 20, 30, 40, 50, 60, 70, 80, 90, 100, 110, 120, 130, 140, 150, 160, 170, 180, 190, 200, 300, 400, 500, 700, 900, 1000, 1100, 1300, and 1500 mg, and any combination thereof. The doses herein are suitable whether for therapeutic or prophylactic administration. Those skilled in the art will appreciate that the foregoing dose levels are provided illustratively, and that higher and lower dose levels can be employed without departing from the spirit and scope of the present invention.

The residence time for the insert in the body cavity, be it buccal, vaginal, or rectal, may range from 1 hour to 2 days. This range includes all values and subranges therebetween, including, for example, 1, 2, 3, 4, 5, 6, 7, 8, 9, 10, 11, 12, 13, 14, 15, 16, 17, 18, 19, 20, 21, 22, 23, 24, 36, and 48 hours, and any combination thereof.

The highest mean plasma concentration, Cₘₐₓ, of clindamycin upon vaginal administration of clindamycin hydrochloride unit dose equivalent to 100 mg clindamycin in contact with a non-degradable hydrogel matrix, measured at one or more of 6, 12, 24, 36, 48, or 72 hours thereafter, may suitably range from 1 to 1000 ng/ml. This range includes all values and subranges therebetween, including 1, 2, 3, 4, 5, 6, 7, 8, 9, 10, 15, 20, 25, 30, 35, 40, 45, 50, 75, 100, 200, 250, 500, 750, and 1000 ng/ml, and any combination thereof.

In one embodiment, the hydrogel matrix may be molded or cast directly into the desired final size and/or shape. In one embodiment, the hydrogel matrix may be polymerized in bulk, then sliced or otherwise trimmed to the desired size and/or shape. The thus-produced hydrogel matrix may then be stored under suitable preserving conditions until further processing.

The hydrogel matrix, either in bulk or in final shape and size, may be purified, for example, in a suitable solvent, such as water, alcohol, ethanol, or a combination thereof, to extract all or a portion of any remaining reactants or uncured polymer from the matrix. In one embodiment, the hydrogel matrix is placed in water or solvent and optionally agitated at a temperature ranging from 10 to 50° C. as appropriate for a time ranging from 1 hour to 2 days as appropriate for extraction and/or purification. The water or solvent may be decanted and the hydrogel matrix may be optionally dried. This process may be repeated as necessary prior to loading of the clindamycin hydrochloride.

The clindamycin hydrochloride and/or any co-administrant or other additive may be simultaneously or consecutively loaded onto the hydrogel matrix. In one embodiment, a loading solution may be prepared by dispersing or dissolving the compound(s) to be loaded in a suitable solvent, for example, water, alcohol, ethanol, or a combination thereof. One or more suitable co-solutes, buffering agents, dispersants, and the like may be added to assist in the loading. The blank hydrogel matrix is placed in the loading solution, with optional agitation, for a time and a temperature sufficient to effect the loading.

In one embodiment, the loading solution is an aqueous solution of clindamycin hydrochloride at a concentration of about 0.1 to 500 M. This range includes all values and subranges therebetween, including, for example, 0.1, 0.2, 0.3, 0.4, 0.5, 1, 2, 3, 4, 5, 10, 11, 12, 13, 14, 15, 20, 40, 60, 80, 100, 200, 300, 400, 500 M clindamycin hydrochloride, and any combination thereof. In one embodiment, the loading solution is a supersaturated solution of clindamycin hydrochloride.

In one embodiment, the loading is carried out at a loading solution temperature ranging from about 5° C. to 60° C. This range includes all values and subranges therebetween, including, for example, 5, 6, 7, 8, 9, 10, 11, 12, 13, 14, 15, 16, 17, 18, 19, 20, 21, 22, 23, 24, 25, 30, 40, 50, and 60° C., and any combination thereof.

In one embodiment, the loading is carried out for a time ranging from about 1 to 48 hours to allow the uptake of the compound(s) to be loaded. This range includes all values and subranges therebetween, including 1, 2, 3, 4, 5, 6, 7, 8, 9, 10, 11, 12, 13, 14, 15, 16, 17, 18, 19, 20, 21, 22, 23, 24, 25, 30, 40, 48 hours, and any combination thereof. The thus loaded hydrogel matrix may then be dried.

The insert may optionally be coated with one or more coatings. Some non-limiting examples of a coating include one or more (co)polymers, soluble (co)polymers, polyvinyl acrylate, methyl cellulose, polyhexylethyl methacrylate, and combinations thereof. The coating may be optionally used, for example, to modify or achieve a particular release profile or other property of the insert.

One embodiment relates to an article of manufacture that may include a packaging material, such as an envelope or sachet, and contained therein an insert which includes at least clindamycin hydrochloride and a hydrogel matrix. The packaging material may include a label which indicates that the insert can be used for ameliorating the symptoms of bacterial vaginosis or other malady by administering the insert.

An insertion system, suitable for inserting the insert into the body cavity, may be used. Such insertion systems may include one or more typical medically and/or commercially acceptable methods for introducing similar items, such as tampons, suppositories, and the like, into a human body cavity, such as the vagina or rectum. Examples of such insertion systems include but are not limited to an applicator, tube, syringe, or the like. The package may be initially sealed, and opened at the time of use. If more than a single dose is present, the package may be resealable by a suitable closure means.

The insert may be used in combination with a retrieval system. Any suitable medically and/or commercially acceptable retrieval system may be used to remove the insert from the body cavity after use so long as it does not interfere with the delivery of the active agent. Some examples of retrieval systems include one or more lines, strings, cords, or ribbons attached to the insert, a molded tab, integral tab extending from the insert, a porous net, porous pouch, knitted tube, or any combination thereof. One example of a suitable retrieval system is disclosed in U.S. Pat. No. 5,269,321, the entire contents of which being hereby incorporated by reference. One or more than one insert may be contained within a retrieval system. The retrieval system may be combined with the insertion system as appropriate.

Any of the packaging material, insertion device, or retrieval device may be irradiated as appropriate.

For optimum loading of clindamycin hydrochloride into the hydrogel matrix, one should consider one or more of the following: weight of loading solution; concentration of clindamycin hydrochloride; batch size; loading temperature; loading time; and/or drying profile of the loaded delivery systems. Given the teachings herein and the knowledge of one of ordinary skill in the art, these may be determined with only routine experimentation.

In addition to clindamycin hydrochloride, one or more additional active ingredients may be optionally co-administered with the insert. The co-administrant may be selected in order to treat one or more of bacterial infections, fungal infections, prophylaxis, e.g., in terminations, dilation and cutterage, ob-gyn examinations, and/or pre-term labor, vaginitis, vaginal candidiasis, genital candidiasis, trichomoniasis, chlamydial infections, and/or gonorrhea.

The co-administrant may be any prophylactic agent or therapeutic agent suitable for vaginal, buccal, or rectal administration. In one embodiment, the co-administrant achieves a local rather than a systemic effect, meaning that the agent functions in the desired beneficial manner without entering the bloodstream. Some local effects may include spermicidal activity, treatment of a vaginal condition or disorder, prevention or treatment of a sexually transmitted disease, and the like. In one embodiment, the co-administrant achieves a local effect in addition to a systemic effect. In one embodiment, the co-administrant achieves a systemic effect. Examples of suitable co-administrants include, without limitation, spermicidal agents, antiviral agents, anti-inflammatory agents, local anesthetic agents, anti-infective agents, antibiotics, antifungal agents, antiparasitic agents, acids, lubricants and mixtures thereof. Some examples of co-administrants are given below:

Spermicidal agents include nonylphenoxypolyethoxy ethanol (sold under the tradename “Nonoxynol-9”), p-diisobutylphenoxy polyethanol (“Octoxynol-9”), benzalkonium chloride, p-methanyl phenylpolyoxyethylene ether (Menfegol), chlorhexidine, polyoxyethylene oxypropylene stearate, ricinoleic acid, glycerol ricinoleate, methyl benzethonium chloride, and mixtures thereof.

Antiviral agents include nucleoside phosphonates and other nucleoside analogs, AICAR (5-amino-4-imidazolecarboxamide ribonucleotide) analogs, glycolytic pathway inhibitors, anionic polymers, and the like, more specifically: antiherpes agents such as acyclovir, famciclovir, foscamet, ganciclovir, idoxuridine, sorivudine, trifluridine, valacyclovir, and vidarabine; and other antiviral agents such as abacavir, adefovir, amantadine, amprenavir, cidofovir, delviridine, 2-deoxyglucose, dextran sulfate, didanosine, efavirenz, indinavir, interferon alpha, lamivudine, nelfinavir, nevirapine, ribavirin, rimantadine, ritonavir, saquinavir, squalamine, stavudine, tipranavir, valganciclovir, zalcitabine, zidovudine, zintevir, and mixtures thereof. Still other antiviral agents are glycerides, particularly monoglycerides, that have antiviral activity. One such agent is monolaurin, the monoglyceride of lauric acid.

Anti-inflammatory agents include corticosteroids, e.g., a lower potency corticosteroid such as hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e. g., hydrocortisone-17,21-diacetate, hydrocortisone-17-acetate-21-butyrate, hydrocortisone-17,21-dibutyrate, etc.), alclometasone, dexamethasone, flumethasone, prednisolone, or methylprednisolone, or a higher potency corticosteroid such as clobetasol propionate, betamethasone benzoate, betamethasone diproprionate, diflorasone diacetate, fluocinonide, mometasone furoate, triamcinolone acetonide, and mixtures thereof.

Local anesthetic agents include acetamidoeugenol, alfadolone acetate, alfaxalone, amucaine, amolanone, amylocaine, benoxinate, benzocaine, betoxycaine, biphenamine, bupivacaine, burethamine, butacaine, butaben, butanilicaine, buthalital, butoxycaine, carticaine, 2-chloroprocaine, cocaethylene, cocaine, cyclomethycaine, dibucaine, dimethisoquin, dimethocaine, diperadon, dyclonine, ecgonidine, ecgonine, ethyl aminobenzoate, ethyl chloride, etidocaine, etoxadrol, β-eucaine, euprocin, fenalcomine, fomocaine, hexobarbital, hexylcaine, hydroxydione, hydroxyprocaine, hydroxytetracaine, isobutyl p-aminobenzoate, ketamine, leucinocaine mesylate, levobupivacaine, levoxadrol, lidocaine, mepivacaine, meprylcaine, metabutoxycaine, methohexital, methyl chloride, midazolam, myrtecaine, naepaine, octacaine, orthocaine, oxethazaine, parethoxycaine, phenacaine, phencyclidine, phenol, piperocaine, piridocaine, polidocanol, pramoxine, prilocaine, procaine, propanidid, propanocaine, proparacaine, propipocaine, propofol, propoxycaine, pseudococaine, pyrrocaine, risocaine, salicyl alcohol, tetracaine, thialbarbital, thimylal, thiobutabarbital, thiopental, tolycaine, trimecaine, zolamine, phenol, and mixtures thereof.

Antibiotic agents include those of the lincomycin family, such as lincomycin; clindamycin, clindamycin salt, clindamycin phosphate, clindamycin acetate, other macrolide, aminoglycoside, and glycopeptide antibiotics such as erythromycin, clarithromycin, azithromycin, streptomycin, gentamicin, tobramycin, amikacin, neomycin, vancomycin, and teicoplanin; antibiotics of the tetracycline family, including tetracycline, chlortetracycline, oxytetracycline, demeclocycline, rolitetracycline, methacycline and doxycycline; and sulfur-based antibiotics, such as the sulfonamides sulfacetamide, sulfabenzamide, sulfadiazine, sulfadoxine, sulfamerazine, sulfamethazine, sulfamethizole, and sulfamethoxazole; streptogramin antibiotics such as quinupristin and dalfopristin; and quinolone antibiotics such as ciprofloxacin, nalidixic acid, ofloxacin, and mixtures thereof.

Antifungal agents include miconazole, terconazole, isoconazole, itraconazole, fenticonazole, fluconazole, ketoconazole, clotrimazole, butoconazole, econazole, metronidazole, clindamycin, 5-fluorouracil, amphotericin B, and mixtures thereof.

Other anti-infective agents include miscellaneous antibacterial agents such as chloramphenicol, spectinomycin, polymyxin B (colistin), and bacitracin, anti-mycobacterials such as such as isoniazid, rifampin, rifabutin, ethambutol, pyrazinamide, ethionamide, aminosalicylic acid, and cycloserine, and antihelminthic agents such as albendazole, oxfendazole, thiabendazole, and mixtures thereof.

The co-administrants may have systemic and/or topical effectiveness against a Candida species, for example against Candida albicans, Candida tropicalis and/or Candida stelloidea, polyene antifungal agent effective against a Candida species, natamycin, nystatin, azole antifungal agent effective against Candida species, clotrimazole, pyrimidine antifungal agent effective against Candida species, flucytozine, ciclopirox olamine, naftifine, terbinafine, haloprogin. Other examples of co-administrants include, tinidazole, amphotericin, capsofungin, griseofulvin, semapimod, itracaonazole, ketoconazole, andiofungilins, voriconazole, acyclovir/aciclovir, famciclovir, tenofovir, zidovudine, azithromycin, and mixtures thereof.

Other optional additives include antioxidants, i.e., agents inhibit oxidation and thus prevent the deterioration of preparations by oxidation. Suitable antioxidants include, by way of example and without limitation, ascorbic acid, ascorbyl palmitate, butylated hydroxyanisole, butylated hydroxytoluene, hypophophorous acid, monothioglycerol, sodium ascorbate, sodium formaldehyde sulfoxylate, sodium metabisulfite, sodium bisulfite, vitamin E and its derivatives, propyl gallate, sulfite derivatives, and others known to those of ordinary skill in the art. Mixtures are possible.

Other optional additives include suitable bacterostats, preservatives, inhibitors, colorants, or the like, such as methyl, ethyl, propyl, and butyl esters of parahydroxybenzoic acid, propyl gallate, sorbic acid and its sodium and potassium salts, propionic acid and its calcium and sodium salts, “Dioxin” (6-acetoxy-2,4-dimethyl-m-dioxane), “Bronopol” (2-bromo-2-nitropropane-1,3-diol) and salicylanilides such as disbromosalicylanilide, tribromosalicylamilides, “Cinaryl” 100 and 200 or “Dowicil” 100 and 200 (Cis isomer of 1-(3-chloroallyl-3,5,7-triaza-1-azanidadamantane chloride), hexachlorophene, sodium benzoate, citric acid, ethylene diaminetetraacetic acid and its alkali metal and alkaline earth metal salts, butyl hydroxyanisole, butyl hydroxytoluene, phenolic compounds such as chloro- and bromocresols and chloro- and bromo-oxylenols, quaternary ammonium compounds like benzalkonium chloride, aromatic alcohols such as phenylethyl alcohol, benzyl alcohol, etc., chlorobutanol, quinoline derivatives such as iodochlorhydroxyquinolin, and the like. Combinations are possible.

Any of the co-administrants may be administered in the form of a salt, ester, amide, prodrug, conjugate, active metabolite, isomer, fragment, analog, or the like, provided that the salt, ester, amide, prodrug, conjugate, active metabolite, isomer, fragment, or analog is pharmaceutically acceptable and is or releases a pharmacologically active agent in the present context. Salts, esters, amides, prodrugs, conjugates, active metabolites, isomers, fragments, and analogs of the agents may be prepared using standard procedures known to those skilled in the art of synthetic organic chemistry and described, for example, by J. March, Advanced Organic Chemistry: Reactions, Mechanisms and Structure, 5th Edition (New York: Wiley-Interscience, 2001).

For example, acid addition salts are prepared from a drug in the form of a free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the co-administrants may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

One or more than one co-administrant and/or additives may be used in the insert.

The amount of the co-administrant(s) in the film will typically range from about 0.01 to about 15% w/w hydrogel matrix. This range includes all values and subranges therebetween, including, for example, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15% w/w hydrogel matrix, and any combination thereof.

In one embodiment, the insert includes butylated hydroxy anisole in an amount ranging from about 0.01 to 0.1% w/w hydrogel matrix. This range includes all values and subranges therebetween, including, for example, 0.01, 0.02, 0.03, 0.04, 0.05, 0.06, 0.07, 0.08, 0.09, 0.1% w/w hydrogel matrix, and any combination thereof.

While the present invention is susceptible to embodiment in many different forms, several embodiments of the invention are described herein in detail. It should be understood, however, that the present disclosure and the embodiments described herein are to be considered as exemplifications of the principles of this invention and are not intended to otherwise limit the invention, as defined by the claims herein.

### EXAMPLES

The following examples are provided for further illustration only, and are not intended to be limiting unless otherwise specified.

### Example 1

A 100 mg clindamycin hydrochloride vaginal insert (CHVI) in accordance with one embodiment was prepared for the treatment of bacterial vaginosis (BV). Using an in vitro microbiological model, the antibacterial efficacy of CHVI was compared to that of two commercially available clindamycin phosphate treatments for BV (CLINDESSE™ and CLEOCIN™). Clindamycin phosphate products were selected as there are no vaginal products on the market at present which employ clindamycin hydrochloride.

In addition, the antibacterial efficacy of clindamycin phosphate loaded on a hydrogel matrix (CVI) was compared to that of the 100 mg clindamycin hydrochloride vaginal insert (CHVI). In this comparison, clindamycin phosphate was loaded on the hydrogel matrix, and 2×50 mg clindamycin phosphate vaginal inserts were used together in one retrieval system.

### Materials

Test microorganism: Bacteroides fragilis NCTC 9344

CLINDESSE™—100 mg clindamycin phosphate units in vaginal cream—comparative example.

CLEOCIN™—100 mg clindamycin phosphate units in melting (degradable) ovule—comparative example.

CHVI—100 mg clindamycin hydrochloride units loaded on a hydrogel matrix—example.

CVI—100 mg clindamycin phosphate units loaded on a hydrogel matrix (2×50 mg units in one retrieval system)—comparative example.

Brain Heart Infusion Broth (BHI)

Neutralizing solution—20 g/l casein plus 10% Tween 80

Bacteriological peptone water

Columbia Blood Agar with 5% Horse Blood

Anaerobic kit—Oxoid AnaeroGen

0.45 μm Filters—Pall GN-6

Gilson pipettes and sterile tips

Sterile spreaders

37°+/−1° C. Incubator

Clindamycin hydrochloride vaginal inserts (CHVI) are composed of a hydrogel polymer with clindamycin hydrochloride dispersed throughout its matrix, contained within a retrieval tape. The hydrogel polymer insert measures 30 mm in length, 10 mm in width and approximately 1.5 mm in thickness. It is rectangular in shape with radiused corners. The components and quantitative composition of the CHVI is given below in Table 1.

TABLE 1
Composition of CHVI

| | Quantity | |
|---|---|---|
| | (mg/unit) | Function |
| Component | | |
| Clindamycin | 109.0 | Active |
| Hydrochloride | | |
| Other ingredients | | |
| Polyethylene glycol | 405.6 | Base polymer |
| 8000 | | |
| 1,2,6 hexanetriol | 8.14 | Cross-linking |
| | | agent |
| Dicyclohexylmethane | 37.2 | Chain extender |
| 4,4′-diisocyanate | | |
| Ferric chloride | 0.04 | Catalyst |
| Butylated hydroxy | 0.25 | Anti-oxidant |
| anisole | | |

The hydrogel polymer is produced by the reaction of molten polyethylene glycol (PEG), Desmodur W (dicyclohexylmethane 4,4′-diisocyanate, DMDI) and hexanetriol (HT) with trace amounts of ferric chloride, which is used as a catalyst. The polymer is poured into molds and, after curing at approximately 95° C. for at least four hours; the polymer is cooled to room temperature. The resulting blocks of polymer are sliced to yield blank slices of the required thickness. The polymer slices may be stored at −20° C. to 25° C. prior to purification.

The blank polymer slices are placed in purified water and agitated at 25° C.±2° C. for approximately 6-8 hours and then the water is decanted. The swollen slices are again placed in purified water and agitated at 25° C.±2° C. for approximately 16-20 hours; the water is then decanted. Water swollen polymer slices are placed in an ethanol:water solution and agitated at 25° C.±2° C. for approximately 6-8 hours. Alternatively purification can take place in water only for 24 hours. The solution is then decanted. The units are dried in a coating pan for approximately 24 hours. The purified polymer slices are stored at −20° C. prior to drug loading.

A drug loading solution is prepared by optionally firstly dispersing the antioxidant, butylated hydroxy anisole (BHA) in water. The clindamycin hydrochloride is dissolved in the resulting solution. Clindamycin hydrochloride used in the CHVI is manufactured by Zhejiang Hisoar Pharmaceuticals and Chemicals Co., Ltd, No 100 Waisha Branch Road, Jiaojiang Taizhou Zhejiang, China, PC 318000. The slices and drug loading solution are agitated at 25° C.±2° C. for approximately 16-24 hours to allow the uptake of drug. Any remaining drug solution is then decanted and the swollen polymer slices are dried with dehumidified air in a coating pan for approximately 24 hours.

### Method

Although no in vitro model will totally mimic the in vivo condition for BV, the in vitro model herein was developed to reflect as far as possible in vivo conditions. These are:

Temperature—the temperature of the healthy vagina is 37° C.+/−1° C. This is the temperature that was used throughout the study.

Nutrient availability—BHI contains all the nutrients required for growth of the test strain,

Incubation conditions—The assay was run under anaerobic conditions for optimal recovery of B. fragilis.

pH—The pH of the broth, BHI, was pH 7.0-7.2. This is within the reported pH range for women suffering from BV (National Guideline for the Management of Bacterial Vaginosis, 2002, Hay PE (www.agum.org.uk/ceg2002), the entire contents of which are hereby incorporated by reference).

Standard 100 ml volumes of broth were used, which is well in excess of the volumes expected in vivo. The large volumes were necessary to allow for repeated sampling and to accommodate CLINDESSE™. CLINDESSE™ is a waxy product which immediately breaks up on mixing. Smaller volumes of broth would have caused sampling problems during filtration.

Several colonies of the test microorganism were inoculated into 10 ml BHI and incubated anaerobically at 37° C. for 24 hours. One ml of the overnight culture suspension was added to 100 ml BHI and mixed on a vortex mixer. One unit of the product under test was added, mixed and immediately one ml was removed and serially diluted in bacteriological peptone water. 0.1 ml of the appropriate dilution was withdrawn, pipetted onto CBA, and spread plates were prepared (0 hour time point).

Further samples were taken at 20 hr, 40 hr and 66 hr. To neutralize the presence of antibiotic carryover, each sample was filtered and rinsed with neutralising solution. At each time point the sample aliquot was added to 50 ml of purified water and passed through a 0.45 μm filter. For CHVI, CVI, and CLEOCIN™, each sample was rinsed with 1×100 ml of neutralizing solution and for CLINDESSE™ 2×100 ml sample volumes were used. After rinsing, each filter was placed onto CBA.

A control was run in parallel as above which contained test microorganism and broth only.

All plates and test samples were incubated anaerobically at 37°+/−1° C. for 48 hours.

### Testing of CHVI and CLINDESSE™

Table 2 below summarizes three independent assays conducted with CHVI and CLINDESSE™ against Bacteroides fragilis.

TABLE 2
Number of cfu/ml

| Time point | CHVI | CLINDESSE ™ | Broth Control |
|---|---|---|---|
| 0 hr | | | |
| Run 1 | 1.69 × 10⁷ | 1.67 × 10⁷ | 2.44 × 10⁷ |
| Run 2 | 1.24 × 10⁷ | 1.31 × 10⁷ | 1.64 × 10⁷ |
| Run 3 | 1.45 × 10⁶ | 1.70 × 10⁶ | 1.65 × 10⁶ |
| 20 hr | | | |
| Run 1 | 2.18 × 10⁶ | 3.13 × 10⁶ | 2.08 × 10⁹ |
| Run 2 | 1.36 × 10⁵ | 2.47 × 10⁶ | 2.57 × 10⁹ |
| Run 3 | 5.0 × 10³ | 5.45 × 10⁵ | 6.5 × 10⁷ |
| 40 hr | | | |
| Run 1 | 1 × 10⁴ | 5.5 × 10⁵ | 4.5 × 10⁹ |
| Run 2 | — | 2.5 × 10⁵ | 1.86 × 10¹⁰ |
| Run 3 | 0 | 5.05 × 10⁴ | 2.0 × 10⁸ |
| 66 hr | | | |
| Run 1 | 8.77 × 10² | 2.89 × 10⁴ | 7.3 × 10¹⁰ |
| Run 2 | 1.14 × 10² | 2.5 × 10⁴ | 5.5 × 10¹⁰ |
| Run 3 | 0 | 7.0 × 10³ | 8.5 × 10⁸ |

As seen in Table 2, 100 mg CHVI unit challenged with an initial inoculum of 10⁷ cfu/ml (runs 1 and 2) achieved a 10⁵ cfu/ml reduction over 66 hours. When challenged with a lower initial inoculum of 10⁶cfu/ml (run 3), no colonies were recovered after 40 hours. For the CLINDESSE™ product, counts reduced by a factor of ten at each time point for both the 10⁶ and 10⁷cfu/ml challenge producing a 10³ cfu/ml reduction over the 66-hour test period. The broth control demonstrates that the microorganisms were not affected over the test period.

### Testing CHVI, CLINDESSE™ and CLEOCIN™

CLEOCIN™ is a commercially available product, which is applied in vivo as 1×100 mg clindamycin phosphate ovule per day for three days. To allow a direct comparison to CHVI and CLINDESSE™, one 100 mg CLEOCIN™ unit was used for each test run. The results are shown in Table 3.

TABLE 3
Number of cfu/ml

| Time | | | | |
|---|---|---|---|---|
| point | CHVI | CLEOCIN ™ | CLINDESSE ™ | Control |
| 0 hr | | | | |
| Run 1 | 7.85 × 10⁵ | 4.95 × 10⁵ | 5.91 × 10⁵ | 9.35 × 10⁵ |
| Run 2 | 3.55 × 10⁵ | 2.15 × 10⁵ | | 2.20 × 10⁵ |
| 20 hr | | | | |
| Run1 | 1.15 × 10⁵ | 4.63 × 10⁵ | 3.47 × 10⁵ | 3.25 × 10⁷ |
| Run 2 | 8.15 × 10⁴ | ~7.5 × 10⁵ | — | 2.35 × 10⁶ |
| 40 hr | | | | |
| Run 1 | 10 | ~7.5 × 10⁴ | ~7.5 × 10⁴ | 7.50 × 10⁷ |
| Run 2 | 0 | 1.45 × 10⁵ | — | 1.35 × 10⁶ |
| 66 hr | | | | |
| Run 1 | 0 | 1.24 × 10⁴ | 2.49 × 10³ | 2.56 × 10¹⁰ |
| Run 2 | 0 | 9.10 × 10⁴ | — | 1.55 × 10⁶ |

As seen in Table 3, CHVI, microbial counts reduced from 10⁵ cfu/ml to 10¹ cfu/ml for run 1 and 0 cfu/ml for run 2 at 40 hours and no colonies were recovered for both runs at 66 hours. For CLINDESSE™, counts reduced by 10² over 66 hours. For CLEOCIN™, no significant reduction was observed. Over 66 hours, only a 10¹ cfu/ml reduction was achieved.

### Testing CVI

Hydrogel polymer units were loaded with clindamycin phosphate (CVI). Two runs were set up with different initial inoculum of 10⁵ and 10⁶ cfu/ml. The results are reported below in Table 4.

TABLE 4
Number of cfu/ml

| Time point | CVI | Control |
|---|---|---|
| 0 hr | | |
| Run 1 | 1.15 × 10⁶ | 1.46 × 10⁶ |
| Run 2 | 2.35 × 10⁵ | 1.75 × 10⁵ |
| 20 hr | | |
| Run1 | 2.96 × 10⁵ | 2.45 × 10⁶ |
| Run 2 | 6.2 × 10⁴ | 4.84 × 10⁸ |
| 40 hr | | |
| Run 1 | ~1.0 × 10⁵ | 5.2 × 10⁹ |
| Run 2 | 6.2 × 10³ | 2.95 × 10⁹ |
| 66 hr | | |
| Run 1 | 2.87 × 10⁴ | 4.9 × 10⁹ |
| Run 2 | 1.87 × 10² | 7.7 × 10⁹ |

As seen in Table 4, CVI challenged with 10⁶ cfu/ml (run 1) achieved a 10² cfu/ml reduction in counts over 66 hours. When tested against an initial inoculum of 10⁵ cfu/ml, counts reduced tenfold at each time point over the 66 hours resulting in a 10³ cfu/ml reduction. The kill rate for CVI is similar to the results observed for CLINDESSE™, but, like CLINDESSE™, was still below that of the CHVI.

The results shown in Tables 1-4 demonstrate the superior efficacy of CHVI to the commercially available products, CLINDESSE™ and CLEOCIN™, and to the clindamycin phosphate loaded on the hydrogel matrix (CVI). In contrast to CHVI, neither CLINDESSE™, CLEOCIN™, nor CVI achieved a kill even after 66 hours. This suggests that CHVI releases more efficiently into the in vitro model and/or that clindamycin hydrochloride is more efficacious than clindamycin phosphate.

Although the level of antibiotic released for CVI is likely to also be above MIC levels, the release profile of CVI in the buffered medium, BHI, is not known. It is clear, however, that regardless of the mechanism, the CVI was inferior to the CHVI in the model, as evidenced by the observed kill rates.

The results demonstrate the microbiological advantage and superior antimicrobial efficacy of CHVI over CVI, CLINDESSE™ and CLEOCIN™. CHVI, when challenged with an initial inoculum of ˜10⁶ cfu/ml of B. fragilis, achieved a kill in 40-66 hours. In comparison, CLINDESSE™ achieved only a 10³ cfu/ml reduction at 66 hrs. CLEOCIN™, challenged with a lower initial inoculum of ˜10⁵ cfu/ml, achieved a tenfold reduction in microbial counts over the 66 hour test period.

Notwithstanding any difficulties correlating in vitro and in vivo data, if CHVI acts in vivo as in the in vitro model as expected, CHVI would provide a better and more efficacious alternative to clindamycin phosphate vaginal products currently on the market.

Assaying clindamycin phosphate units loaded in the polymer (CVI) allowed a direct comparison of the two drugs (clindamycin phosphate and clindamycin hydrochloride) loaded in the same polymer. The results show that CVI was not as effective in the in vitro model as CHVI. For CVI, counts reduced by only 10²-10³ cfu/ml over the 66 hours, whereas CHVI produced a kill in 40-66 hours. The results for CVI were similar to the results observed for CLINDESSE™.

An in vitro static drug release experiment was set up for CHVI in BHI media. It showed that ˜80% of the drug was released in 18 hours. As the drug remains in the model and is not lost, levels would have been well above the reported MIC of 2 μg/ml for clindamycin sensitive B. fragilis (Lorian V. “Antibiotics in Laboratory Medicine”, 4^{th} Edition Williams & Wilkens, 1996, the entire contents of which being hereby incorporated by reference). As such, it is expected that the superior results observed in vitro for CHVI would extend to in vivo applications.

### Example 2

### Drug Release and Stability

One batch of CHVI was prepared and tested for stability. Table 5 provides 12-month stability data for CHVI at 25° C. and 40° C. storage conditions.

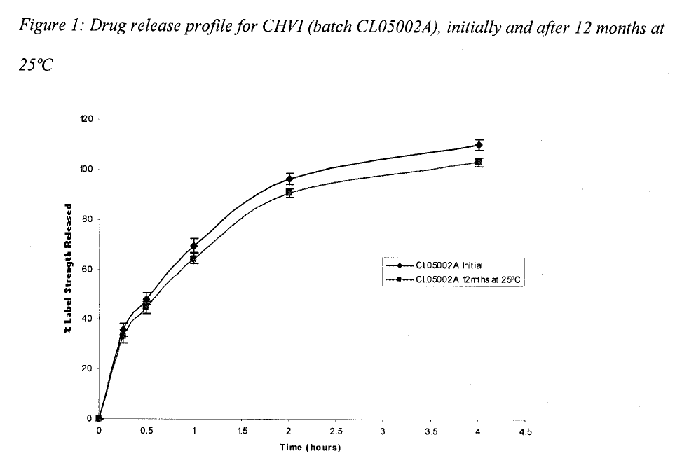

FIG. 1 provides drug release profiles for CHVI stored at 25° C. initially and after 12 months.

TABLE 5
Stability data for CHVI 100 mg LS, 3, 6 and 12 months at 25° C. and 40° C.

| | | 3 months | | 6 months | | 12 months | |
|---|---|---|---|---|---|---|---|
| TEST | Initial | 25° C. | 40° C. | 25° C. | 40° C. | 25° C. | 40° C. |
| Clindamycin Potency % LS* | 100.03 | 101.4 | 101.8 | 102.6 | 101.6 | 101.0 | Not Tested |
| | Pass | Pass | Pass | Pass | Pass | Pass | |

Drug Release (% LS release)

| | | | | | | | |
|---|---|---|---|---|---|---|---|
| 0.25 hours | 35.8% | 34.2% | 33.4% | 35.2% | 34.6% | 33.1% | Not Tested |
| 0.5 hours | 48.2% | 46.2% | 45.6% | 47.3% | 46.8% | 44.8% | |
| 1 hour | 69.5% | 66.4% | 66.7% | 68.1% | 67.8% | 64.8% | |
| 2 hours | 96.4% | 92.1% | 94.0% | 93.9% | 93.8% | 90.6% | |
| 4 hours | 110.2% | 104.4% | 108.5% | 106.8% | 106.3% | 103.5% | |
| | Pass | Pass | Pass | Pass | Pass | Pass | |
| Loss on Drying (% w/w) | 0.60% | 0.61% | 0.65% | 0.61% | 0.65% | 0.53% | Not Tested |
| | Pass | Pass | Pass | Pass | Pass | Pass | |
| Butylated Hydroxyanisole | 0.05% | 0.05% | 0.05% | 0.05% | 0.05% | 0.05% | Not Tested |
| (% w/w) | Pass | Pass | Pass | Pass | Pass | Pass | |

*LS = label strength

The real time stability data demonstrates that the CHVI is stable when stored at 25° C. and 40° C. for up to twelve months. The drug release profile is unchanged. BHA content is unchanged.

A stability study was also carried out on CVI, clindamycin phosphate on hydrogel with BHA (butylated hydroxyl anisole) and citric acid (present as antioxidant and loading co-solute, respectively). It was found that the CVI was not stable at 25° C. or 40° C. for one month (data not shown).

Accordingly, the CHVI is more stable than the CVI.

What is claimed is:

1. An insert, comprising:
a non-degradable hydrogel matrix; and
clindamycin hydrochloride in contact with the matrix;
wherein said insert is suitable for mammalian intravaginal, buccal, or intrarectal use.

2. The insert of claim 1, wherein the insert is suitable for intravaginal use.

3. The insert of claim 1, wherein the insert is suitable for buccal use.

4. The insert of claim 1, wherein the insert is suitable for intrarectal use.

5. The insert of claim 1, wherein the clindamycin hydrochloride is present in an amount ranging from about 5 to 75% w/w hydrogel matrix.

6. The insert of claim 1, wherein the clindamycin hydrochloride is present in an amount ranging from about 15 to 30% w/w hydrogel matrix.

7. The insert of claim 1, wherein the clindamycin hydrochloride is present in an amount ranging from about 20 to 25% w/w hydrogel matrix.

8. The insert of claim 1, wherein the insert comprises an amount of clindamycin hydrochloride equivalent to 100 mg unit dose of clindamycin.

9. The insert of claim 1, further comprising an antioxidant.

10. The insert of claim 1, further comprising butylated hydroxy anisole in an amount ranging from about 0.01 to 0.1% w/w hydrogel matrix.

11. The insert of claim 1, further comprising butylated hydroxyl anisole in an amount ranging from about 0.03 to 0.07% w/w hydrogel matrix.

12. The insert of claim 1, further comprising a device adapted to retrieve the insert from a vagina or rectum.

13. The insert of claim 1, further comprising and in contact with a device adapted to retrieve the insert form a vagina or rectum and selected from the group consisting of line, cord, ribbon, molded tab, integral tab, porous net, porous pouch, knitted tube, and a combination thereof.

14. The insert of claim 1, further comprising a device adapted to insert the insert into a vagina or rectum.

15. The insert of claim 1, further comprising and in contact with a device selected from the group consisting of an applicator, syringe, tube, stick, and a combination thereof.

16. The insert of claim 1, which is suitable for human intravaginal, buccal, or intrarectal use.

17. The insert of claim 1, which is suitable for non-human intravaginal, buccal, or intrarectal use.

18. The insert of claim 1, further comprising one or more co-administrants.

19. The insert of claim 1, wherein the hydrogel matrix comprises a crosslinked polyethylene glycol polymer.

20. The insert of claim 1, wherein the hydrogel matrix comprises a crosslinked polymer of polyethylene glycol and urethane.

21. The insert of claim 1, wherein the hydrogel matrix comprises a crosslinked polymer having a gel:sol ratio of 75:25 or more.

22. The insert of claim 1, wherein the insert exhibits a drug release profile substantially as given in FIG. 1.

23. A method, comprising contacting a mammalian vagina, buccal cavity, or rectum with the insert of claim 1.

24. The method of claim 23, wherein said vagina, buccal cavity, or rectum is that of a human female.

25. The method of claim 23, wherein said contacting is carried out to treat or prevent bacterial vaginosis in a human female.

26. The method of claim 23, wherein said contacting is carried out to treat or prevent bacterial vaginosis in a human female known or suspected to have bacterial vaginosis.

27. The method of claim 23, wherein said contacting is carried out to treat or prevent bacterial vaginosis in a human female at risk for bacterial vaginosis.

28. The method of claim 23, wherein said contacting is continuous for period of time ranging from one hour to two days.

29. The method of claim 23, wherein said contacting is repeated about one to four times daily.

30. The method of claim 23, wherein said contacting is repeated about one to four times daily over a time period ranging from one day to one year.

31. The method of claim 23, wherein the contacting is sufficient to inhibit at least one microorganism in a vagina.

32. The method of claim 31, wherein the microorganism is a fungus.

33. The method of claim 31, wherein the microorganism is a bacterium.

34. The method of claim 31, wherein the microorganism is a yeast.

35. The method of claim 31, wherein the microorganism is a mold.

36. A method, comprising contacting clindamycin hydrochloride with a non-degradable hydrogel matrix.

37. The method of claim 36, wherein said non-degradable hydrogel matrix is swollen during said contacting.

38. The method of claim 36, wherein said clindamycin hydrochloride is present in an aqueous or ethanolic solution during said contacting.

39. The method of claim 36, wherein said clindamycin hydrochloride is present in a solution having a clindamycin hydrochloride concentration ranging from about 0.1 to about 20 M.

40. The method of claim 36, further comprising contacting the non-degradable hydrogel matrix with at least one co-administrant.

41. The method of claim 36, further comprising contacting the non-degradable hydrogel matrix with butylated hydroxyl anisole.

42. The method of claim 36, further comprising, after said contacting, drying the hydrogel matrix.

43. A package, comprising:
the insert of claim 1; and
at least one packaging material surrounding the insert.

44. A retrievable device, comprising:
the insert of claim 1; and
a device in contact with the insert and adapted to retrieve the insert from a vagina or rectum.

45. The retrievable device of claim 44, wherein the device is selected from the group consisting of line, cord, ribbon, molded tab, integral tab, porous net, porous pouch, knitted tube, and a combination thereof.

46. The retrievable device of claim 44, further comprising a device adapted to insert the insert into a vagina or rectum.

47. An insertable device, comprising:
the insert of claim 1; and
a device in contact with the insert and adapted to insert the insert into a vagina or rectum.

48. The insertable device of claim 47, wherein the device is selected from the group consisting of an applicator, syringe, tube, stick, and a combination thereof.
